(19) United States
(12) Patent Application Publication
(10) Pub. No.: US 2005/0123956 A1
(43) Pub. Date: Jun. 9, 2005

(54) Methods for modifying DNA for microarray analysis

(75) Inventors: John E. Blume, Danville, CA (US); Yanxiang Cao, Mt. View, CA (US); Glenn H. McGall, Palo Alto, CA (US); Kyle B. Cole, Stanford, CA (US); Fredrick C. Christians, Los Altos Hills, CA (US); Kai Wu, Mt. View, CA (US); Linda Hsie, San Jose, CA (US); Charles G. Miyada, San Jose, CA (US); Anthony D. Barone, San Jose, CA (US); Vivi Truong, Mt. View, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/951,983

(22) Filed: Sep. 27, 2004

Related U.S. Application Data
(60) Provisional application No. 60/506,697, filed on Sep. 25, 2003.
(60) Provisional application No. 60/512,569, filed on Oct. 15, 2003.
(60) Provisional application No. 60/547,915, filed on Feb. 25, 2004.
(60) Provisional application No. 60/514,872, filed on Oct. 28, 2003.
(60) Provisional application No. 60/512,301, filed on Oct. 16, 2003.

(51) Int. Cl. C12Q 1/68

(52) U.S. Cl. 435/6

(57) ABSTRACT

In one aspect of the invention, methods and compositions are provided for fragmenting nucleic acid samples. Fragmented nucleic acid samples may be used for hybridization with microarrays.

### PRIORITY CLAIM

The present application claims priority to U.S. Provisional Application Ser. No. 60/506,697 filed on Sep. 25, 2003, U.S. Provisional Application Ser. No. 60/512,569 filed on Oct. 15, 2003, U.S. Provisional Application Ser. No. 60/512,301 filed on Oct. 16, 2003, U.S. Provisional Application Ser. No. 60/514,872 filed on Oct. 28, 2003 and U.S. Provisional Application Ser. No. 60/547,915 filed on Feb. 25, 2004. All cited patent applications are incorporated herein by reference in its entirety for all purposes.

### BACKGROUND OF THE INVENTION

Nucleic acid sample preparation methods have greatly transformed laboratory research that utilize molecular biology and recombinant DNA techniques and have also impacted the fields of diagnostics, forensics, nucleic acid analysis and gene expression monitoring, to name a few. There remains a need in the art for methods for reproducibly and efficiently fragmenting nucleic acids used for hybridization on oligonucleotide arrays.

### SUMMARY OF THE INVENTION

In one aspect of the invention, methods and compositions (including reagent kits) are provided for fragmenting nucleic acid samples. In preferred embodiments, the methods and compositions are used to fragment DNA samples for gene expression (transcript) monitoring and for genotyping assays.

In a preferred embodiment, RNA transcript samples are used as templates for reverse transcription to synthesize single strand cDNA (ss-cDNA) or double strand cDNA (ds-cDNA). Methods for synthesizing cDNA are well known in the art. In another embodiment, resulting cDNA may be used as templates for in vitro transcription reactions to synthesize cRNA. The cRNAs are then used as template for another cDNA synthesis reaction as described in Whole Transcript Assay (WTA) or small sample WTA (sWTA) protocols described for example in U.S. patent application Ser. No. 10/917,643. In a preferred embodiment, a modified precursor nucleotide Deoxyuracil (dUTP) is incorporated into cDNA during first and/or second-strand cDNA synthesis. cDNA synthesis using the precursor nucleotides dATP, dCTP, dGTP and dUTP in place of dTTP results in DNA complementary to the template where Thymine is replaced by Uracil. Other modified nucleic acid precursors can also be used, such as dITP and 8-OH dGTP.

The glycosylase substrate precursors dUTP, dITP and 8OHdGTP when incorporated into DNA generate the glycosylase substrate bases Uracil, Hypoxanthine and 8-OH guanine, respectively. In a preferred embodiment, the DNA glycosylase is Uracil DNA Glycosylase (UDG). Uracil in DNA is recognized specifically by UDG and released from DNA, generating an abrasic site. Several agents are known which cleaves the phosphodiester bonds in nucleic acids at abrasic sites. Agents that cleaves 5′ to the phosphate moiety and generate 3′terminus with a free 3′OH are the enzyme with endonuclease activity, such as endonuclease IV and endonuclease V from E. Coli and AP endonuclease such as Human ApeI endonuclease, and the like. In a combined reaction, UDG removes the Uracil base and the endonuclease removes the apyridimic site leaving a 3′ hydroxyl available for labeling.

Alternatively, in another embodiment E. coli endonuclease V is used for fragmenting ds or ss-cDNA without the addition of UDG. Endonuclease V from E. Coli recognizes several modified bases in DNA including Uracil, Hypoxanthine (ionisine). Endonuclease V has been shown to fragment DNA without requiring the presence of Uracil in the substrate for DNA cleavage.

The fragmentation process produces DNA fragments within a certain range of length that can subsequently be labeled. In a preferred embodiment, the average size of fragments obtained is at least 10, 20, 30, 40, 50, 60, 70, 80, 100 or 200 nucleotides.

In one embodiment, the fragment size is controlled by the amount of dUTP that is incorporated in during cDNA synthesis. In a preferred embodiment the ratio of dTTP to dUTP is selected to generate DNA fragments of a predetermined size range. For example, dUTP concentration can be decreased in order to increase the size of the DNA fragments. In a preferred embodiment, a ratio of 1 dU to 3 dT is used (see FIG. 4) After fragments have been end-labeled, DNA fragments may be hybridized to a microarray of probes. Example of microarray that my be used for analysis are available from Affymetrix and include for example HG-U133A2.0 array. In a preferred embodiment the arrays may have probes that target at least 50%, 60%, 70%, 80%, 90% or all the exons of at least 500, 1000 or 10000 transcripts.

The reagent kits of the invention typically include some combination of the reagents useful for the methods of the invention. For example, one reagent kit includes dUTP, Ape1 endonuclease and a suitable microarray. Optionally, the reagent kit may include, for example, labeling reagents, reverse transcriptase, etc.

In another aspect of the invention, dsDNA is cut into many small fragments using a combination of multiple enzymes with a short recognition sequence, e.g. a “4-cutter.” 4-cutters restriction enzymes allow the cleavage of target DNA at many potential sites, resulting in a collection of random DNA fragments. For example, DNA may be cut using multiple restriction enzymes including Sau3AI, AluI, RsaI, AciI, BfaI, MboI, FatI, HinP1 I, HpaII, MspI, TaqI, Bst UI, HaeIII, PhoI, MseI and/or DpnII.

In another aspect of the invention, the methods comprise means of controlling the length of DNA fragments during the synthesis of the target nucleic acid. For example, length of DNA fragments may be controlled for during the synthesis of the first or second cDNA strand.

Reverse transcriptase is an RNA-dependent DNA polymerase and will synthesize a first-strand cDNA complementary to an RNA template, using a mixture of four dNTPs, under the appropriate conditions and for a sufficient amount of time for the enzymatic processes to take place. Reverse transcriptase are generally derived from RNA-containing viruses such as Avian Myeloblastosis Virus (AMV) or Maloney Murine Leukemia Virus (MMLV).

In addition to polymerase activity, RT possesses an RNase H activity that degrades the RNA in an RNA/DNA hybrid resulting in shorter cDNA synthesis in vitro (Berger S. et al. (1983) Biochemistry, 22: 2365-2372). For longer cDNA, the RNase H domain of RT can be mutated to reduce or eliminate RNase H activity while maintaining mRNA-directed DNA polymerase activity. Removal of RT RNase H activity improves the efficiency of cDNA synthesis from mRNA catalyzed by RT (Kotewicz M. et al. (1988) Nucleic Acids Res., 16:265-277). In a preferred embodiment, reverse transcriptase having a RNase H activity is used.

Reverse transcriptase has a tendency to pause during cDNA synthesis resulting in the generation of truncated products (Harrison,G. et al. (1998) Nucleic Acids Res., 26:3433-3442). This pausing is due in part to the secondary structure of RNA. Performing cDNA synthesis at reaction temperatures that begin to melt the secondary structure of mRNA (>55° C.) helps to alleviate this problem (Myers T. and Gelfand D.(1991) Biochemistry, 30: 7661-7666).

Short cDNA fragments (50 to 200 bps) may be synthesized by selecting a reverse transcriptase having an RNase H activity such as MMLV-RT that has not been modified to increase its thermal stability and under sub-optimal conditions. Sub-optimal conditions may include modifying the incubation temperature; decreasing the incubation time below 60 min., heat inactivating the enzyme prior use and modifying the nucleotide concentration. In one embodiment, nucleotide analogs such as dideoxyNTPs (ddNTPs) are incorporated in the reverse transcriptase mix for the first strand cDNA synthesis, blocking the polymerization by the reverse transcriptase.

The ratio primer to template and the specificity of the primers are important parameters for controlling the length of the newly synthesized strand. In a preferred embodiment, short cDNA fragments are synthesized by increasing the primer to template concentration. In another embodiment, short cDNA strands may be synthesized by using non-specific primers such as random hexamers.

Yet, in another embodiment, reverse transcriptase may be mutagenized in order to favor short cDNA strands synthesis.

The second strand cDNA synthesis is catalyzed by the Klenow fragment of the DNA polymerase I. In a preferred embodiment, dideoxyNTPs (ddNTPs) are incorporated in the reverse transcriptase mix for the second strand cDNA synthesis. The presence of ddNTPs blocks polymerization by the Klenow Fragment. Since the incorporation of ddNTP rather than dNTP is a random event, the reaction will produce DNA fragments varying in length. In a preferred embodiment, the ratio of dNTP to ddNTP is selected to generate DNA fragments of a predetermined size range. For example, DNA fragments sized may range from 50 to 200 bases.

In a preferred embodiment the multiple copies of cDNA generated by the disclosed methods are analyzed by hybridization to an array of probes. The nucleic acids generated by the methods may be analyzed by hybridization to nucleic acid arrays. Those of skill in the art will appreciate that an enormous number of array designs are suitable for the practice of this invention. High density arrays may be used for a variety of applications, including, for example, gene expression analysis, genotyping and variant detection. Array based methods for monitoring gene expression are disclosed and discussed in detail in U.S. Pat. Nos. 5,800,992, 5,871,928, 5,925,525, 6,040,138 and PCT Application WO92/10588 (published on Jun. 25, 1992). Suitable arrays are available, for example, from Affymetrix, Inc. (Santa Clara, Calif.).

### BRIEF DESCRIPTION OF THE DRAWINGS

The accompanying drawings, which form a part of this specification, illustrate embodiments of the invention, and together with the description, serve to explain the principles of the invention:

FIG. 1 is a schematic drawing of a preferred embodiment employing DNA endonuclease fragmentation and terminal labeling of double-stranded cDNA. dUTP can be incorporated into first strand cDNA by reverse transcriptase and into second-strand cDNA by DNA polymerase I (1-2). Uracil DNA-glycosylase (UDG) specifically removes uracil bases leaving apyrdimic sites that are recognized and excised by endonuclease IV (Endo IV) leaving 3′-OH that can be labeled using terminal transferase (TdT) and Affymetrix DNA Labeling Reagent (DLR1a)(3-4).

FIG. 2 is a schematic drawing of a preferred embodiment employing DNA endonuclease fragmentation and terminal labeling of single-stranded cDNA. (1) dUTP is incorporated into first strand cDNA by reverse transcriptase (MMLV or SuperScript II). (2) RNA templates is removed by hydrolysis with NaOH or with RNase H. (3) Uracil DNA-glycosylase (UDG) specifically removes uracil bases leaving apyridimic sites (4) that are excised by endonuclease IV (Endo IV) leaving 3′-OH that can be (5) labeled using terminal transferase (TdT) and Affymetrix DNA Labeling Reagent (DLR1a).

FIG. 3 compares the performance of separate and simultaneous fragmentation/labeling. Combined UDG/Endo IV fragmentation and TdT end-labeling. Note nearly equivalent fragmentation and labeling efficiency of combined reaction. Lane 1: HiLo molecular weight marker, lane 2: unfragmented ds-cDNA, lane 3: ds-cDNA fragmented with UDG/Endo IV and labeled with TdT in a separate reaction, lane 4: previous sample gel-shifted with streptavidin, lane 5: ds-cDNA fragmented with UDG/Endo IV and simultaneously labeled with TdT, lane 6: previous sample gel-shifted with streptavidin.

FIG. 4 shows that the average fragment size is controlled by dUTP concentration. ds-cDNA was synthesized using varying amounts of dUTP in the first and second-strand synthesis reactions. ds-cDNA was fragmented and labeled following the DEFT protocol. Note that average fragment size (denoted by red star) increases as dUTP concentration decreases (lanes 4-7).

FIG. 5 shows the fragment size distribution determined by BioAnalyzer. Note that the average fragment size of ds cDNA containing 1 dU:3 dT in both the sense and antisense strands is 78 nt after DEFT fragmentation. (5 B)

FIG. 6 shows optimization of DEFT labeling reaction. dUTP was incorporated into only the sense stand, only the anti-sense strand or both strands of double-stranded cDNA. The cDNA was fragmented with UDG/Endo IV and end-labeled with TdT and DLR1a in a combined reaction or separately. 1:3 and 1:4 ratios of dUTP:dTTP were also tested. Columns 1 and 2 (yellow) represent the array performance of DNase I fragmented ds-cDNA. Column three: dUTP incorporated into antisense strand, fragmented and labeled simultaneously. Column four: dU in both sense and anti-sense strands, fragmentation and labeling in separate reactions. Column five: Same as column four with lower amount of Endo IV (2 U/ug). Column 6: cDNA with dU only in anti-sense strand, ratio 1 dU:3 dT. Column 7: cDNA with dU only in anti-sense strand, ratio 1 dU:4 dT. Column 8: cDNA with dU in both strands at 1 dU:3 dT.

### DETAILED DESCRIPTION OF THE INVENTION

A. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

B. Definitions

The term “array” as used herein refers to an intentionally created collection of molecules which can be prepared either synthetically or biosynthetically. The molecules in the array can be identical or different from each other. The array can assume a variety of formats,for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “fragmentation” refers to the breaking of nucleic acid molecules into smaller nucleic acid fragments. In certain embodiments, the size of the fragments generated during fragmentation can be controlled such that the size of fragments is distributed about a certain predetermined nucleic acid length.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization conditions” as used herein will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage disequilibrium” or sometimes refer by allelic association as used herein refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

C. The Nucleic Acid Fragmentation Methods and Compositions

In one aspect of the invention, methods and compositions are provided for fragmenting a nucleic acid target such as DNA and RNA. In a preferred embodiment, RNA transcripts samples are used as template for a reverse transcription reaction to synthesize cDNAs. The cDNAs may be fragmented and hybridized with a microarray or alternatively, the cDNAs may be used as templates for cDNA synthesis. Methods for synthesizing cDNA are well known in the art. Sample preparation for Whole Transcript Assays are described, for example, in U.S. patent application Ser. No. 10/917,643 which is incorporated herein by reference. Both single-stranded and double-stranded DNA targets may be fragmented. The methods of the invention are particularly suitable for use with arrays that interrogate a large portion of the transcripts, such as tiling arrays, all exon arrays, and alternative splicing arrays.

One of skill in the art would appreciate that the methods and compositions are useful for fragmenting nucleic acids in many applications in addition to assays that measures RNA transcripts. For example, the methods and compositions are also useful for genotyping assays such as the Whole Genome Sampling Assays (WGSA, Affymetrix, Santa Clara) for use with commercially available 10 K or 100 K SNP genotyping arrays.

While the methods of the invention has broad applications and are not limited to any particular detection methods, they are particularly suitable for detecting a large number of, such as more than 1000, 5000, 10,000, 50,000 different transcript features.

Fragmentation of nucleic acids comprises breaking nucleic acid molecules into smaller fragments. Fragmentation of nucleic acid may be desirable to optimize the size of nucleic acid molecules for certain reactions and destroy their three dimensional structure. For example, fragmented nucleic acids may be used for more efficient hybridization of target DNA to nucleic acid probes than non-fragmented DNA. According to a preferred embodiment, before hybridization to a microarray, target nucleic acid should be fragmented to sizes ranging from 50 to 200 bases long to improve target specificity and sensitivity. In a more preferred embodiment, the average size of fragments obtained is at least 10, 20, 30, 40, 50, 60, 70, 80, 100 or 200 nucleotides.

Labeling may be performed before or after fragmentation using any suitable methods. Labeling methods are well known in the art and are discussed in numerous references including those incorporated by reference.

In one preferred methods, the products of the fragmentation methods are substrates for 3′ end labeling with Affymetrix biotinylated DNA Labeling Reagent (DLR—Affymetrix, Santa Clara, Calif., USA) and terminal deoxynucleotidyl transferase (TdT). Labeled dNTPs can be incorporated this way onto the 3′-OH end of DNA in a template independent reaction. See also, U.S. patent application Ser. Nos. 60/545,417, 60/542,933, 10/452,519 and 10/617,992.

In some preferred embodiments, the methods include of fragmentation employed post cDNA synthesis. Enzymatic fragmentation includes for example digestion with DNase I that generates random distribution of fragments. When fragmenting with DNase I, it may be difficult to control the rate and therefore the extent of fragmentation, potentially giving variable assay performance results. In preferred embodiments, methods that allow for improved control of the rate of fragmentation are disclosed.

In preferred embodiments, robust and efficient methods for fragmentation that are compatible with TdT and DLR end-labeling are disclosed. The disclosed methods may be used, for example, for fragmenting and labeling nucleic acid sample prior to hybridization to an array of probes.

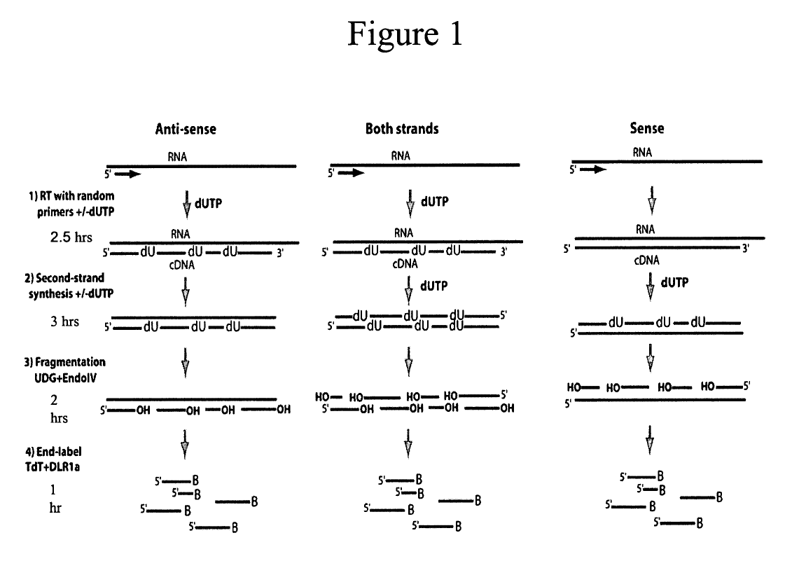

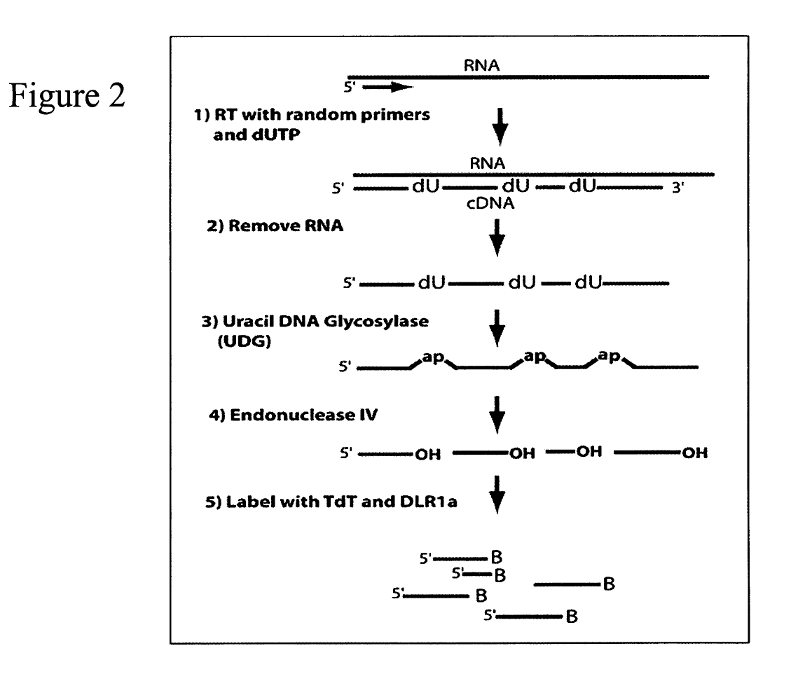

In a preferred embodiment, RNA transcript samples are used as templates for reverse transcription to synthesize single strand cDNA (ss-cDNA) or double strand cDNA (ds-cDNA). Methods for synthesizing cDNA are well known in the art. In another embodiment, resulting cDNA may be used as templates for in vitro transcription reactions to synthesize cRNA. The cRNAs are then used as template for another cDNA synthesis reaction as described in Whole Transcript Assay (WTA) or small sample WTA (sWTA) protocols described for example in U.S. patent application Ser. No. 10/917,643. In a preferred embodiment, a modified precursor nucleotide Deoxyuracil (dUTP) is incorporated into cDNA during first and/or second-strand cDNA synthesis as shown in FIGS. 1 and 2. dUTP is a base sugar phosphate comprising the base Uracil and a sugar phosphate moiety. cDNA synthesis using the precursor nucleotides dATP, dCTP, dGTP and dUTP in place of dTTP results in DNA complementary to the template where Thymine is replaced by Uracil. It will be appreciated by those skilled in the art that other modified nucleic acid precursors can also be used, such as dITP and 8-OH dGTP. The glycosylase substrate precursors dUTP, dITP and 8OHdGTP when incorporated into DNA generate the glycosylase substrate bases Uracil, Hypoxanthine and 8-OH guanine, respectively. In a preferred embodiment, the DNA glycosylase is Uracil DNA Glycosylase (UDG). Uracil in DNA is recognized specifically by UDG and released from DNA, generating an abrasic site. Several agents are known which cleaves the phosphodiester bonds in nucleic acids at abrasic sites. Agents that cleaves 5′ to the phosphate moiety and generate 3′terminus with a free 3′OH are the enzyme with endonuclease activity, such as endonuclease IV and endonuclease V from E. Coli and AP endonuclease such as Human ApeI endonuclease, and the like. In a combined reaction, UDG removes the Uracil base and the endonuclease removes the apyridimic site leaving a 3′ hydroxyl available for labeling.

Alternatively, in another embodiment E. coli endonuclease V is used for fragmenting ds or ss-cDNA without the addition of UDG. Endonuclease V from E. Coli recognizes several modified bases in DNA including Uracil, Hypoxanthine (ionisine). Endonuclease V has been shown to fragment DNA without requiring the presence of Uracil in the substrate for DNA cleavage.

The fragmentation process produces DNA fragments within a certain range of length that can subsequently be labeled. In a preferred embodiment, the average size of fragments obtained is at least 10, 20, 30, 40, 50, 60, 70, 80, 100 or 200 nucleotides.

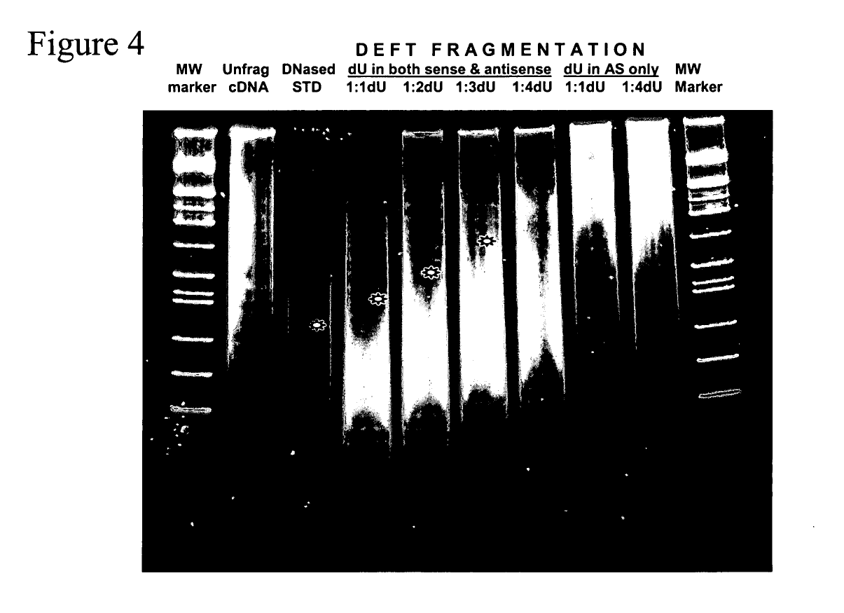

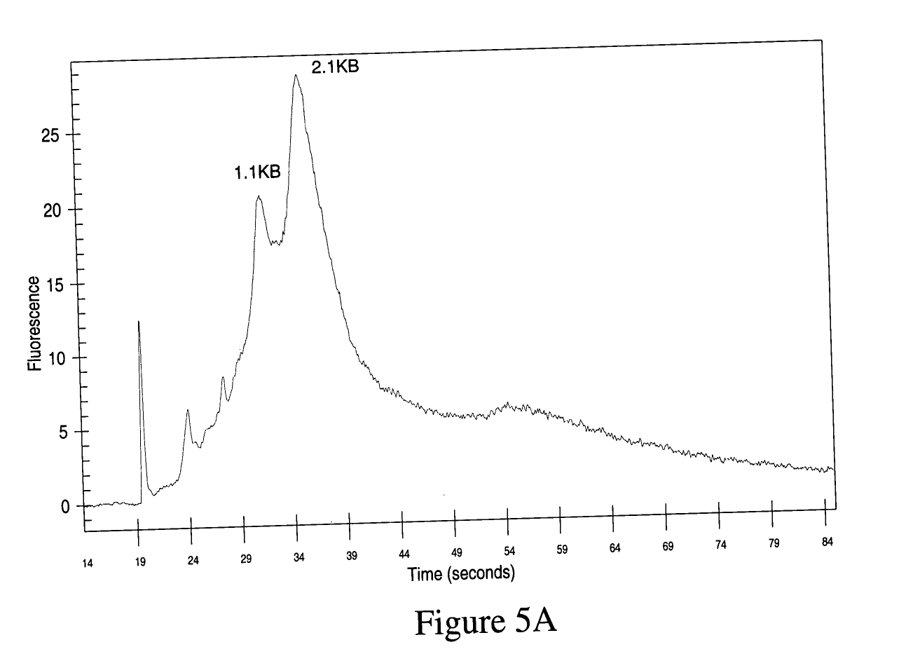

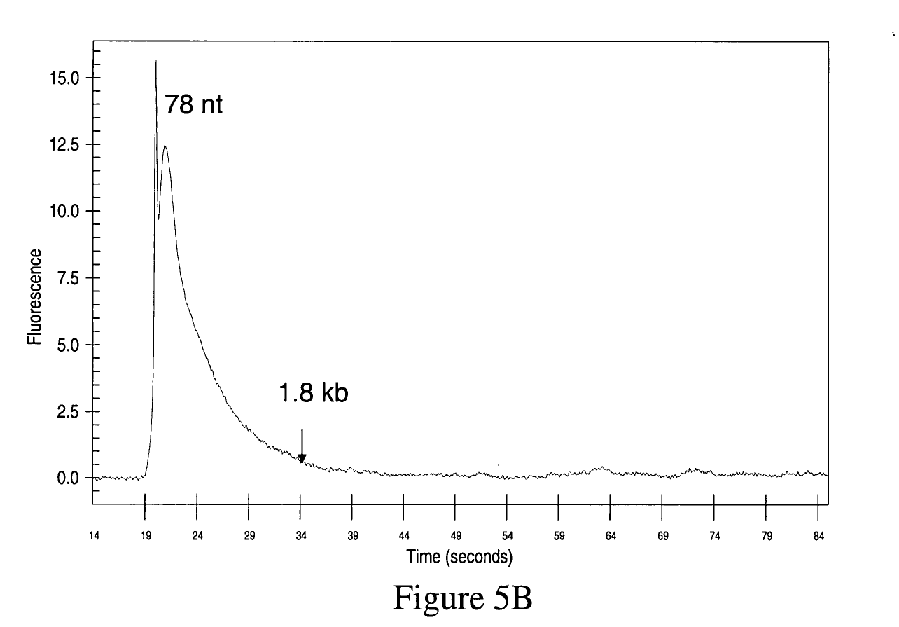

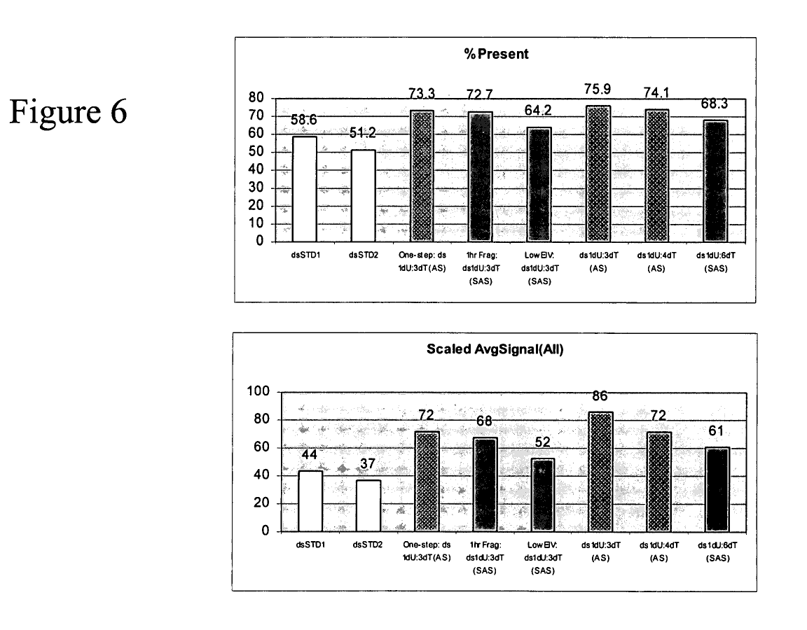

In one embodiment, the fragment size is controlled by the amount of dUTP that is incorporated in during cDNA synthesis. In a preferred embodiment the ratio of dTTP to dUTP is selected to generate DNA fragments of a predetermined size range. For example, dUTP concentration can be decreased in order to increase the size of the DNA fragments. In a preferred embodiment, a ratio of 1 dU to 3 dT is used (see FIG. 4)

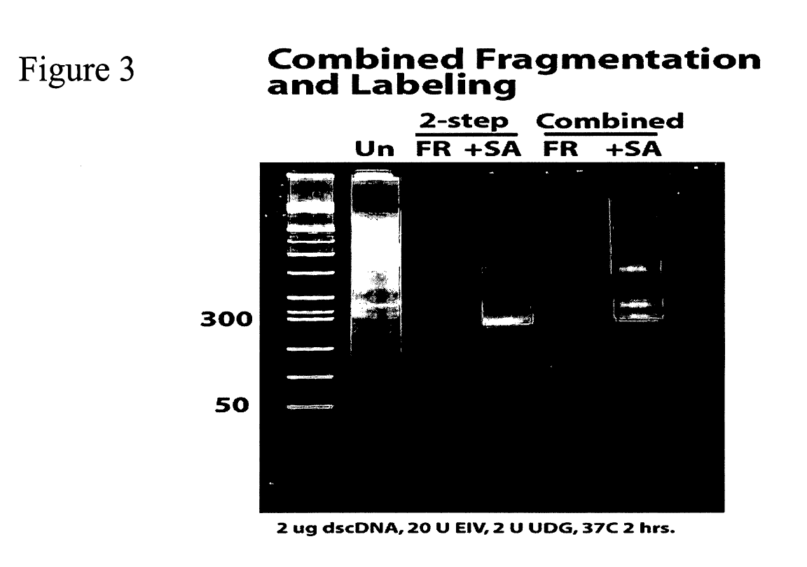

In one embodiment, fragmentation and labeling of ss-cDNA or ds-cDNA is a two step process. Yet in a preferred embodiment, fragmentation and labeling of ss-cDNA or ds-cDNA is performed at the same time. See FIG. 3.

After fragments have been end-labeled, DNA fragments may be hybridized to a microarray of probes. Examples of microarrays that may be used for analysis are available from Affymetrix and include for example HG-U133A2.0 array. In a preferred embodiment the arrays may have probes that target at least 50%, 60%, 70%, 80%, 90% or all the exons of at least 500, 1000 or 10000 transcripts.

The following are detailed protocols as non limiting examples to illustrate the some embodiments of the invention.

| Components | Volume | | Final Concentration |
|---|---|---|---|
| 5X TdT Reaction Buffer | 14 | μl | 1X |
| 25 mM CoCl2 | 14 | μl | 5 mM |
| Endo IV (20 U/μl) | 3.5 | μl | 70 U/3 μg cDNA |
| cDNA template (1.5-5 μg) | 30 | μl | |
| Nuclease-free H₂O | X | μl | |
| Total Volume | 70 | μl | |

1. Incubate the reaction at 37° C. for 120 minutes
2. Inactive Endo IV at 65° C. for 15 minutes
   DEFT Protocol (DNA Endonuclease Fragmentation and Terminal Labeling)
   Two-Step Protocol for ss-cDNA:

1. UDG/Endonuclease IV reaction
1.5 μg sscDNA
4.5 μl 10× Endonuclease IV Buffer
4.5 μl UDG 2U/μl
4.5 μl Endonuclease IV 20U/μl
×μl H2O
Total Volume: 45 μl
Incubate at 37° C. for 1-2 hrs. Enzyme is heat inactivated at 93° C. for 1 min.

2. Labeling Reaction
16 μl 5× Roche TdT Buffer
16 μl 25 mM CoCl2
5 μl TDT 400 U/μl
1.2 μl DLR 5 mM
× μl H2O
Total Volume: 80 μl.
Incubate at 37° C. for 1 h.
   Two-Step Protocol for ds-cDNA:

1. UDG/Endonuclease IV reaction
9 μg dscDNA
4.5 μl 10× Endonuclease IV Buffer
3 μl UDG 2U/μl
3 μl Endonuclease IV 20U/μl
× μl H2O
Total Volume: 45 μl
Incubate at 37° C. for 1-2 hrs. Enzyme is heat inactivated at 93° C. for 1 min.

2. Labeling Reaction
16 μl 5× Roche TdT Buffer
16 μl 25 mM CoCl2
5 μl TDT 400 U/μl
1.2 μl DLR 5 mM
× μl H2O
Total Volume: 80 μl.
Incubate at 37° C. for 1 h.
   Use of Four-Cutter Restriction Enzymes

Restriction enzymes (or restriction endonucleases) are produced in bacteria, presumably to degrade foreign DNA. Methylation differences between the bacterium's genomic DNA and the foreign DNA protect the genomic DNA from cleavage (Venetianer, P. and A. Kiss (1981) In: Gene Amplification and Analysis, Volume 1: Restriction Endonucleases, J. Chirikjian, ed. (Elsevier North Holland, Inc.) 209-215).

Restriction enzymes bind at recognition sequences. Recognition sequences are typically 4 to 6 bases long, but may be longer. The majority of the restriction enzymes cleave double-stranded DNA (dsDNA) at a restriction site, which may or may not be located within the recognition sequence. At each restriction site, one phosphodiester bond from each of the strands in the dsDNA is hydrolyzed to form hydroxyl and phosphate groups. The cleaved sites, one on each DNA strand, may be opposite each other forming two blunt-ended dsDNA fragments, or may occur at different locations resulting in fragments with protruding unpaired bases called sticky ends (Blakesley, R. (1981) In: Gene Amplification and Analysis, Volume 1: Restriction Endonucleases, J. Chirikjian, ed. (Elsevier North Holland, Inc.) 1-34).

In a preferred embodiment, dsDNA is cut into many small fragments using a combination of multiple enzymes with a short recognition sequence, e.g. a “4-cutter.” 4-cutters restriction enzymes allow the cleavage of target DNA at many potential sites, resulting in a collection of random DNA fragments. For example, DNA may be cut using multiple restriction enzymes including Sau3AI, AluI, RsaI, AciI, BfaI, MboI, FatI, HinP1 I, HpaII, MspI, TaqI, Bst UI, HaeIII, PhoI, MseI and/or DpnII.

In another aspect of the invention, the methods comprise means of controlling the length of DNA fragments during the synthesis of the target nucleic acid. For example, length of DNA fragments may be controlled for during the synthesis of the first or second cDNA strand.

Reverse transcriptase is an RNA-dependent DNA polymerase and will synthesize a first-strand cDNA complementary to an RNA template, using a mixture of four dNTPs, under the appropriate conditions and for a sufficient amount of time for the enzymatic processes to take place. Reverse transcriptase are generally derived from RNA-containing viruses such as Avian Myeloblastosis Virus (AMV) or Maloney Murine Leukemia Virus (MMLV).

In addition to polymerase activity, RT possesses an RNase H activity that degrades the RNA in an RNA/DNA hybrid resulting in shorter cDNA synthesis in vitro (Berger S. et al. (1983) Biochemistry, 22: 2365-2372). For longer cDNA, the RNase H domain of RT can be mutated to reduce or eliminate RNase H activity while maintaining mRNA-directed DNA polymerase activity. Removal of RT RNase H activity improves the efficiency of cDNA synthesis from mRNA catalyzed by RT (Kotewicz M. et al. (1988) Nucleic Acids Res., 16:265-277). In a preferred embodiment, reverse transcriptase having a RNase H activity is used.

Reverse transcriptase has a tendency to pause during cDNA synthesis resulting in the generation of truncated products (Harrison,G. et al. (1998) Nucleic Acids Res., 26:3433-3442). This pausing is due in part to the secondary structure of RNA. Performing cDNA synthesis at reaction temperatures that begin to melt the secondary structure of mRNA (>55° C.) helps to alleviate this problem (Myers T. and Gelfand D.(1991) Biochemistry, 30: 7661-7666).

Short cDNA fragments (50 to 200 bps) may be synthesized by selecting a reverse transcriptase having an RNase H activity such as MMLV-RT that has not been modified to increase its thermal stability and under sub-optimal conditions. Sub-optimal conditions may include modifying the incubation temperature; decreasing the incubation time below 60 min., heat inactivating the enzyme prior use and modifying the nucleotide concentration. In one embodiment, nucleotide analogs such as dideoxyNTPs (ddNTPs) are incorporated in the reverse transcriptase mix for the first strand cDNA synthesis, blocking the polymerization by the reverse transcriptase.

The ratio primer to template and the specificity of the primers are important parameters for controlling the length of the newly synthesized strand. In a preferred embodiment, short cDNA fragments are synthesized by increasing the primer to template concentration. In another embodiment, short cDNA strands may be synthesized by using non-specific primers such as random hexamers.

Yet, in another embodiment, reverse transcriptase may be mutagenized in order to favor short cDNA strands synthesis.

The second strand cDNA synthesis is catalyzed by the Klenow fragment of the DNA polymerase I. In a preferred embodiment, dideoxyNTPs (ddNTPs) are incorporated in the reverse transcriptase mix for the second strand cDNA synthesis. The presence of ddNTPs blocks polymerization by the Klenow Fragment. Since the incorporation of ddNTP rather than dNTP is a random event, the reaction will produce DNA fragments varying in length. In a preferred embodiment, the ratio of dNTP to ddNTP is selected to generate DNA fragments of a predetermined size range. For example, DNA fragments sized may range from 50 to 200 bases.

In a preferred embodiment the multiple copies of cDNA generated by the disclosed methods are analyzed by hybridization to an array of probes. The nucleic acids generated by the methods may be analyzed by hybridization to nucleic acid arrays. Those of skill in the art will appreciate that an enormous number of array designs are suitable for the practice of this invention. High density arrays may be used for a variety of applications, including, for example, gene expression analysis, genotyping and variant detection. Array based methods for monitoring gene expression are disclosed and discussed in detail in U.S. Pat. Nos. 5,800,992, 5,871,928, 5,925,525, 6,040,138 and PCT Application WO92/10588 (published on Jun. 25, 1992). Suitable arrays are available, for example, from Affymetrix, Inc. (Santa Clara, Calif.).

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

1. A method for analyzing a nucleic acid sample containing RNA, the method comprising:
obtaining a nucleic acid sample containing RNA;
synthesizing cDNA in presence of one modified DNA precursor nucleotide that is a substrate for a DNA glycosylase;
cleaving the cDNA at the abrasic sites with an endonuclease as to generate a plurality of fragments with free 3′-OH terminus;
labeling the fragments with biotin in a reaction comprising TdT;
hybridizing labeled fragments with a microarray of probes; and
analyzing hybridization pattern.

2. A method according to claim 1 wherein the modified nucleic acid precursor is dUTP.

3. A method according to claim 2 wherein the step of cleaving comprises excising the modified base by means of an Uracil DNA glycosylase so as to generate an abrasic site and cleaving at the abrasic sites by means of an endonuclease.

4. The method according to claim 3 wherein the endonuclease is endonuclease IV.

5. The method according to claim 3 wherein the endonuclease is endonuclease ApeI.

6. The method according to claim 1 wherein the cDNA is cleaved at the abrasic sites by means of an endonuclease V.

7. A method according to claim 1 wherein the modified precursor nucleotide partially replaces one of the normal precursor nucleotides.

8. A method according to claim 7 wherein the ratio dUTP to dTTP is 1 to 3.

9. A method according to claim 1 wherein fragments size range from at least 10 bps to 200 bps.

10. A method according to claim 1 wherein the cleaving and the labeling steps are simultaneous.

11. A method according to claim 1 wherein the nucleic acid sample is mRNA.

12. A method according to claim 1 wherein the cDNA is ss-cDNA.

13. A method according to claim 1 wherein the cDNA is ds-cDNA.

14. A method according to claim 1 wherein dUTP is incorporated into the ss-cDNA during reverse transcription.

15. A method according to claim 1 wherein dUTP is incorporated into the ds-cDNA during second strand cDNA synthesis.

16. A method according to claim 15 wherein dUTP is incorporated in a single or in both strands of ds-cDNA.

17. A method for analyzing a nucleic acid sample, the method comprising:
obtaining a nucleic acid sample containing ds DNA;
digesting ds-DNA with a mixture of four-cutter restriction enzymes generating a plurality of fragments;
labeling the fragments with biotin in a reaction comprising TdT;
hybridizing labeled fragments with a microarray of probes; and
analyzing hybridization pattern.

18. A method according to claim 17 wherein the nucleic acid sample is DNA.

19. A method according to claim 17 wherein the nucleic acid sample is RNA.

20. A method for analyzing a nucleic acid sample containing RNA, the method comprising:
obtaining an RNA from nucleic acid sample;
synthesizing a first strand cDNA using a reverse transcriptase under conditions that promote short strand synthesis;
synthesizing a second strand cDNA using Klenow fragment;
labeling the fragments with biotin in a reaction comprising TdT;
hybridizing labeled fragments with a microarray of probes; and
analyzing hybridization pattern.

21. A method according to claim 20 wherein the reverse transcriptase is MMLV-RT.

22. A method according to claim 20 wherein the step of synthesizing the first strand cDNA is under saturating primer concentration.

23. A method according to claim 20 wherein the step of synthesizing the first strand cDNA is in presence of ddNTPs in the reaction mix.

24. A method for analyzing a nucleic acid sample containing RNA, the method comprising:
obtaining a nucleic acid sample containing RNA;
synthesizing a first strand cDNA using a reverse transcriptase;
synthesizing a second strand cDNA using Klenow fragment under conditions that promote short strand synthesis;
labeling the fragments with biotin in a reaction comprising TdT;
hybridizing labeled fragments with a microarray of probes; and
analyzing hybridization pattern.

25. A method according to claim 24 wherein the step of synthesizing second strand cDNA is in presence of ddNTPs in the reaction mix.
